(19) United States
(12) Patent Application Publication
(10) Pub. No.: US 2026/0345332 A1
(43) Pub. Date: Nov. 5, 2026

(54) Devices For Mechanical Securement To Hard Tissue With Complex Geometry

(71) Applicant: The Trustees of Columbia University in the City of New York, New York, NY (US)

(72) Inventors: Elias Issa, Hillsdale, NJ (US); Ashley Green, Stamford, CT (US)

(21) Appl. No.: 19/657,571

(22) Filed: Apr. 24, 2026

Related U.S. Application Data
(60) Provisional application No. 63/797,662, filed on Apr. 30, 2025.

(51) Int. Cl. A61F 2/30; A61F 2/36; A61F 2/38; A61F 2/40

(52) U.S. Cl. CPC A61F 2/30749; A61F 2/36; A61F 2/389; A61F 2/4081

(57) ABSTRACT

Disclosed herein are prosthesis for securement by force closure to an end portion of a bone that includes both positive and negative curvature, comprising a first enclosure component that at least partially conforms to and engages a first portion of a side surface of the bone and a first portion of an end surface of the bone; a second enclosure component that at least partially conforms to and engages a second portion of a side surface of the bone and a second portion of an end surface of the bone, the first portion of the side surface of the bone being on a substantially opposing side of the bone as the second portion of the side surface of the bone, and wherein the first enclosure component is configured to be coupled to the second enclosure component while said first and second enclosure components are positioned on the bone in order to provide mechanical securement of the prosthesis to the bone. Also provided herein are methods for securing a prothesis according to the present disclosure to an end of a bone, comprising using a jig or guide to determine a cut location on the end of the bone; cutting the bone at the cut location to form a cut section of the bone; engaging at least a portion of the cut section of the bone with the first enclosure portion of the prosthesis; installing the second enclosure portion of the prosthesis onto the end of the bone; and, coupling the first enclosure portion and the second enclosure portion, thereby securing the prosthesis to the end of the bone.

### CROSS-REFERENCE TO RELATED APPLICATIONS

The present application claims the benefit of priority to U.S. Provisional App. No. 63/797,662, filed Apr. 30, 2025, the entire contents of which are incorporated herein by reference.

### TECHNICAL FIELD

The present disclosure pertains to bone prostheses.

### BACKGROUND

High demand for joint replacements. Artificial joint replacements are a common procedure for a large fraction of the human population and commonly include the shoulder, knee, and hip joints. With life expectancy increasing and the desire of patients to be physically active in the later years of life, continued refinements of artificial joint replacement technology will be key to improving patient quality of life whether through increases in mechanical strength, implant longevity, or more efficient surgical implant procedures and fewer revision surgeries.

Causes and indications. In most cases, joint replacement is indicated by arthritic processes in the cartilaginous joint interface (i.e., where one bone end interfaces and articulates with another bone). This is created by wear and tear at the joint which is exacerbated by intense physical activities such as sports and the physiological conditions of obesity and advancing age.

Securement of joint replacements. In a joint replacement, the implanted prosthetics that replace and augment the damaged bone end are secured to the remaining healthy bone via bolts/screws, large penetrating shafts also called stems, and/or cement bonding agents.

Drawbacks of securement by penetrating bolts or stems. Securement by penetrating screws, bolts, or stems is a dominant method for securing implants at joints. However, drilling the otherwise healthy bone in the case of screws or reaming the bone in the case of bolts or stems increases surgical time as well as adding complexity in planning the exact position and angle of bone removal. Furthermore, even after penetrating the bone, fixation is not immediately achieved as time is needed post-surgery for osseointegration to provide adequate mechanical hold between implant and bone. Thus, patients have to wait for weeks to months to completely resume normal activity levels in order to avoid any setbacks immediately post-surgery when implant osseointegration is incomplete.

Mechanically speaking, the elongated axis of most bolts and shafts creates a lever arm for transmitting and magnifying joint forces and thus increases stress shielding of bone in certain directions. As a result of mechanical stress and micromotion, this can lead to a macrophage-induced inflammatory response which tilts the balance in favor of bone degradation (osteolysis) and loosening of the penetrating implant, a failure mode called aseptic loosening, accompanied by patients experiencing enough pain to reduce activity or warranting a revision surgery which carries increased mortality rates. Aseptic loosening is a common concern even for total knee and hip replacements with low complication rates overall (<10%). If a revision surgical procedure is required, then there is significantly less bone available for new penetrating securement as the bone was previously drilled or reamed for the initial penetrating implant. With younger patients seeking joint replacements to maintain high activity levels and/or with patients living longer in an aging population, initial implants may not have the extended lifetime necessary for covering the lifetime use of the individual, and thus the difficulty of performing revision surgeries in conventional implant designs will remain a salient, tangible long-term concern for many patients in joint replacement surgery.

Mitigating the degree of penetration of bolts or stems. Alternative applications/technologies have pursued the goal of sparing bone to a larger degree than in prevailing standard implants. These alternatives still utilize some form of penetrating bolt or shaft but the degree of penetration is reduced. Unfortunately, using limited penetration depth has proven not fully tenable leading to downstream drawbacks. In neck preserving hip resurfacing, if a short bolt is used, then the relatively thin neck of the femur experiences more stress shielding, and the risk of neck fracture increases. Using stronger materials like metals in hip resurfacing for more component strength leads to other complications like leeching of metal particles which then can trigger inflammation leading to aseptic loosening as well as higher circulating levels of metals in the bloodstream which may have systemic effects. Compared to the thick femur portion used in hip replacements, the glenoid bone in shoulder replacements is fairly thin, and often the glenoid is compromised by the arthritic joint process, further reducing the amount of bone available for securement. Hence, bolts are used but given their limited purchase in the glenoid bone, failure rates in shoulder replacements tend to be higher than for standard knee and hip replacements that rely on the thicker femur.

Mitigating degree of penetration via alternative securement by cement bonding agents. Emulating aspects of osseointegration, bonding agents allow for a broad contact surface by filling in gaps between bone and the device (typically a metal stem) with the benefit of immediate fixation compared to penetrating screws that require time for bone growth for osseointegration. However, bonding agents: (1) increase surgical time and complexity, (2) cements are ultimately still foreign bodies being only mildly biocompatible such that they do favor a slightly pro-inflammatory state, (3) cements are susceptible to degradation over time in the biological milieu, and (4) cements can become a nidus for infection or inflammation. Critically, should a revision be needed, cemented implants are difficult to revise as cement is retained on bone, and this forms a further bacterial nidus for relapse events of any new implant.

### SUMMARY

Disclosed herein are prosthesis for securement by force closure to an end portion of a bone that includes both positive and negative curvature, comprising a first enclosure component that at least partially conforms to and engages a first portion of a side surface of the bone and a first portion of an end surface of the bone; a second enclosure component that at least partially conforms to and engages a second portion of a side surface of the bone and a second portion of an end surface of the bone, the first portion of the side surface of the bone being on a substantially opposing side of the bone as the second portion of the side surface of the bone, and wherein the first enclosure component is configured to be coupled to the second enclosure component while said first and second enclosure components are positioned on the bone in order to provide mechanical securement of the prosthesis to the bone.

Also provided herein are methods for securing a prothesis according to the present disclosure to an end of a bone, comprising using a jig or guide to determine a cut location on the end of the bone; cutting the bone at the cut location to form a cut section of the bone; engaging at least a portion of the cut section of the bone with the first enclosure portion of the prosthesis; installing the second enclosure portion of the prosthesis onto the end of the bone; and, coupling the first enclosure portion and the second enclosure portion, thereby securing the prosthesis to the end of the bone.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1-29 provide non-limiting illustrations of the disclosed technology.

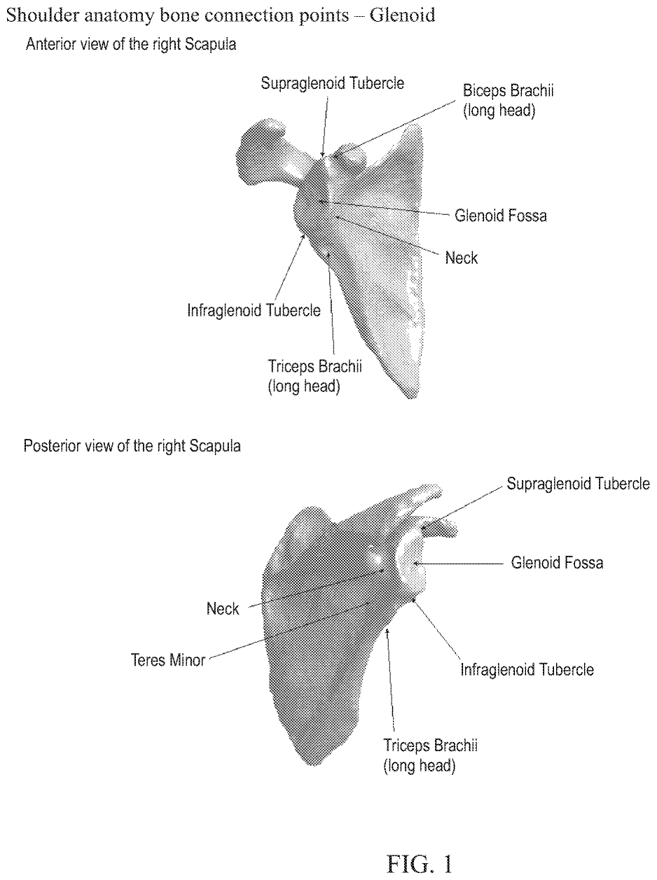

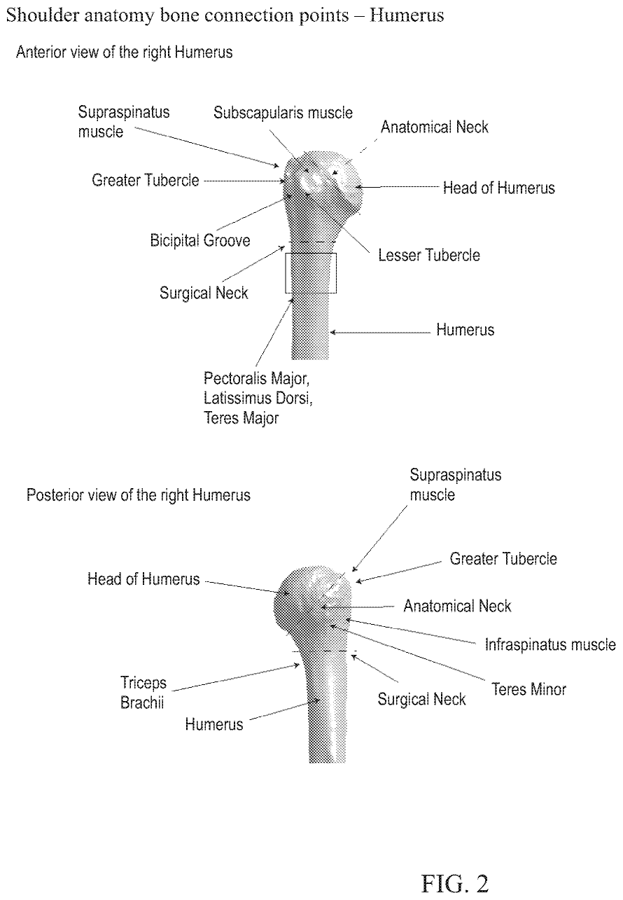

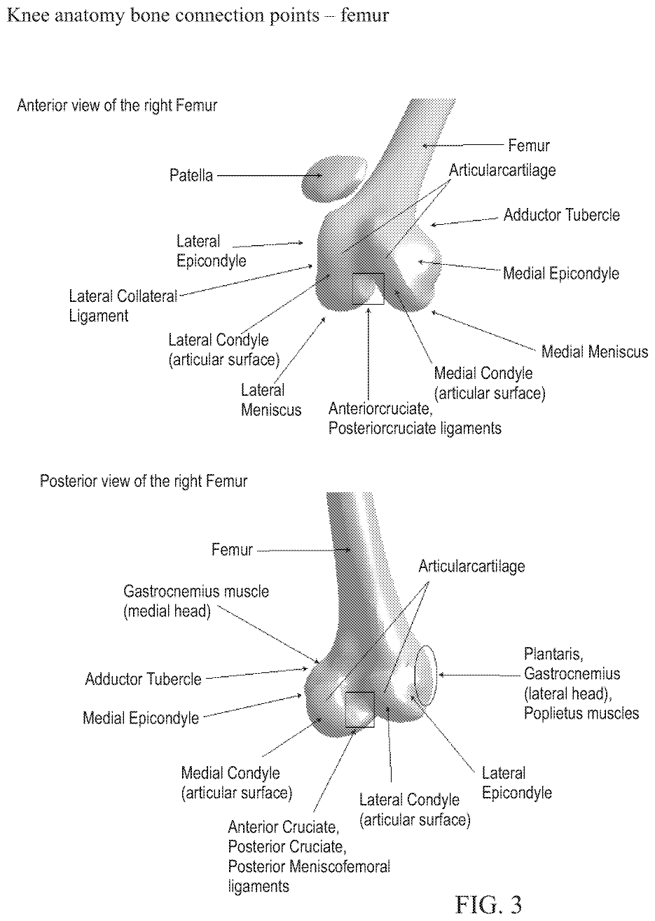

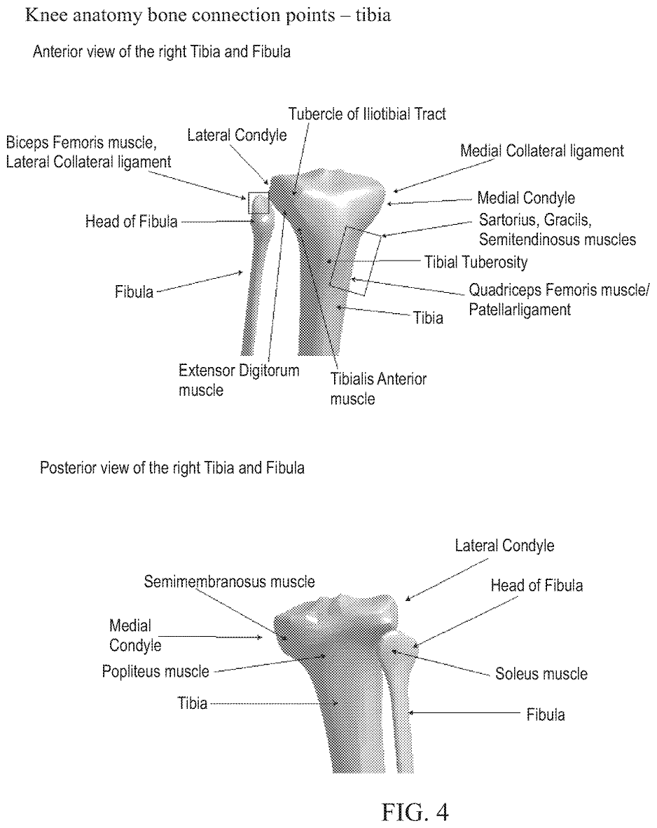

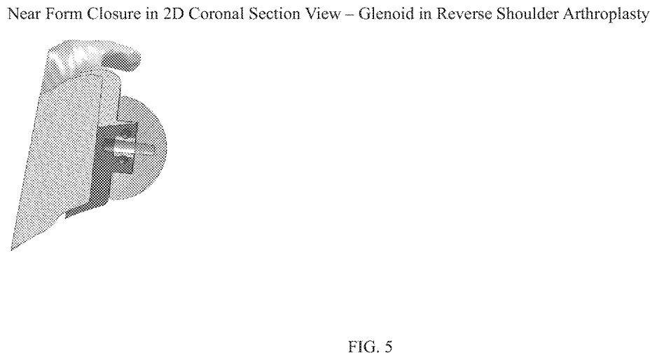

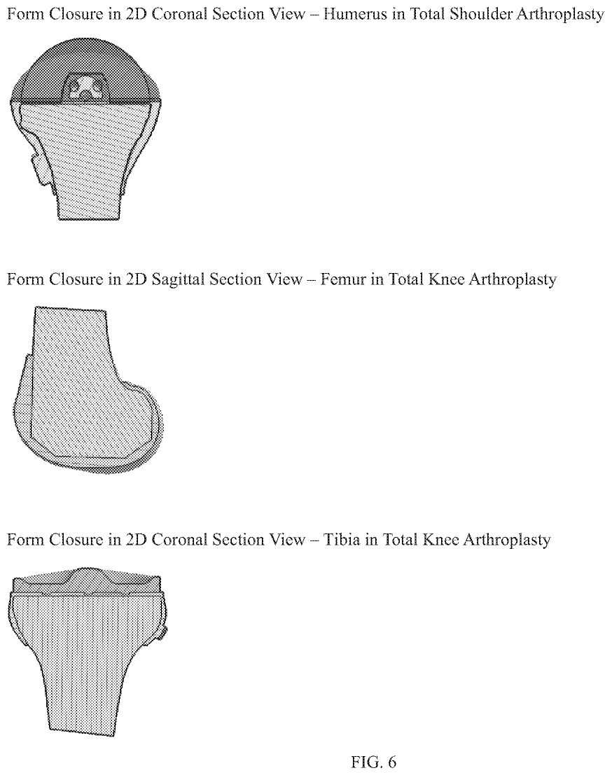

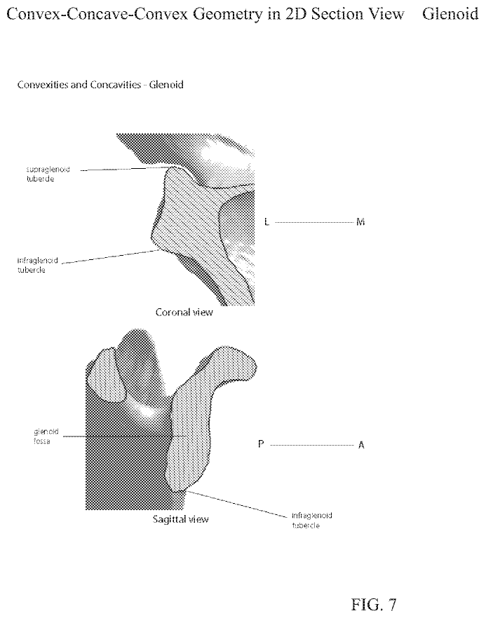

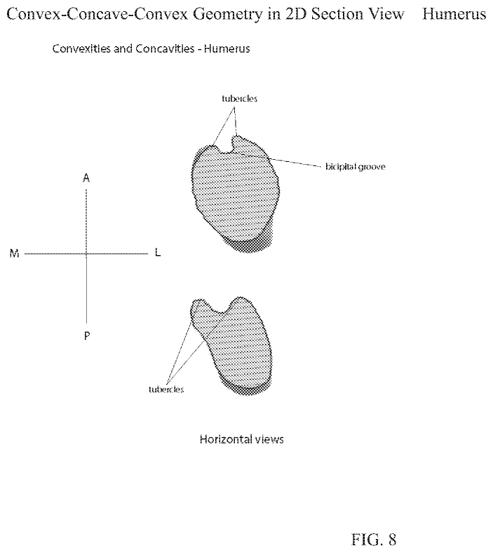

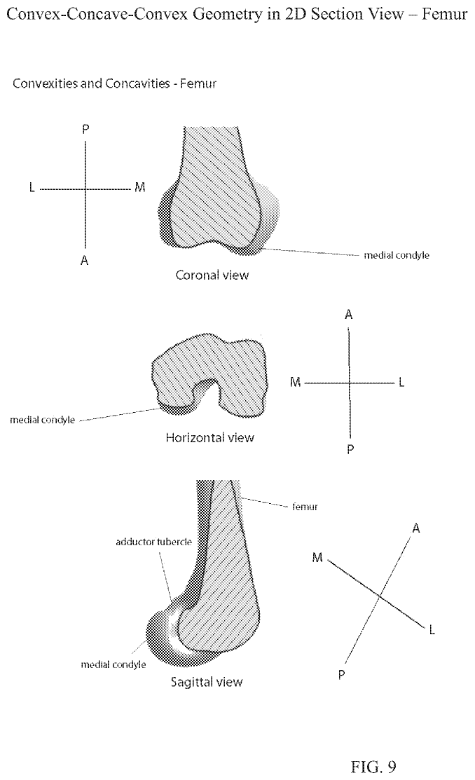

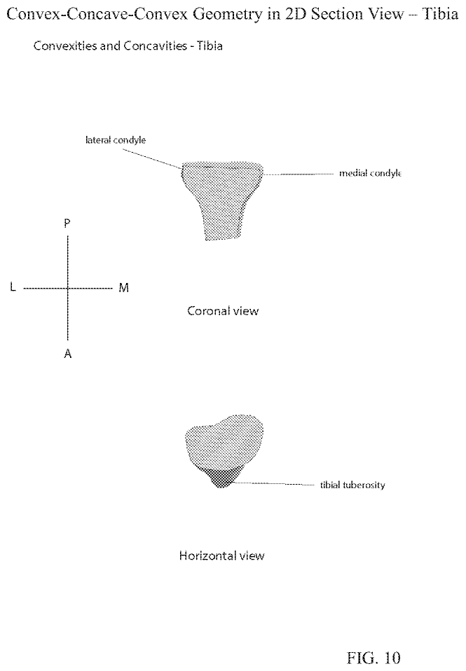

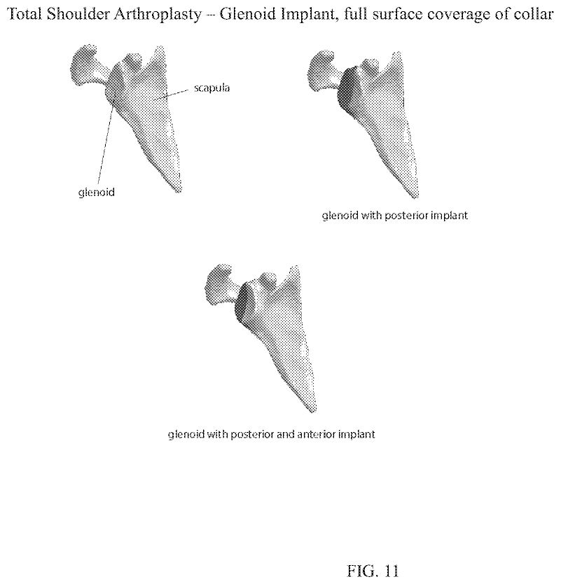

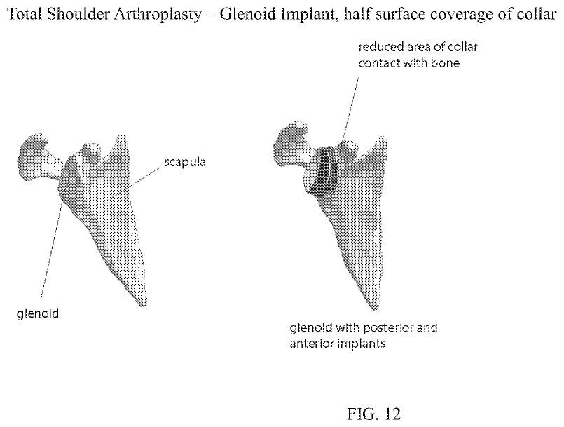

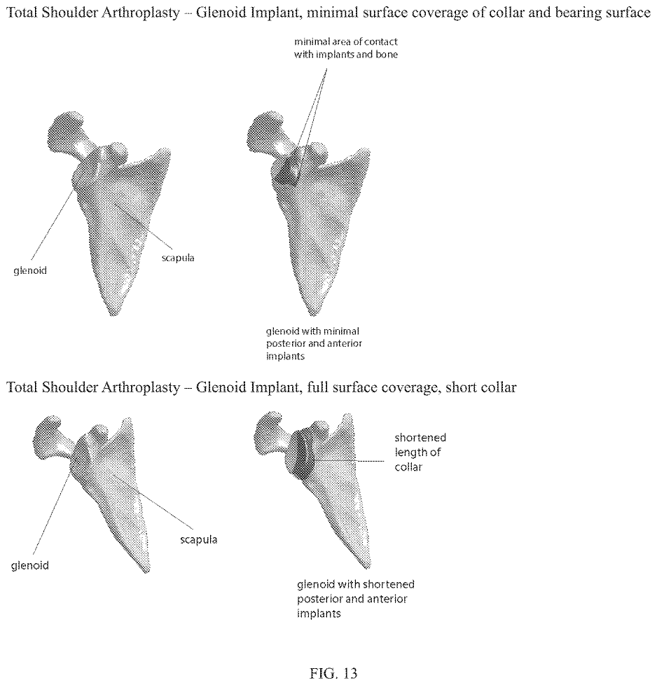

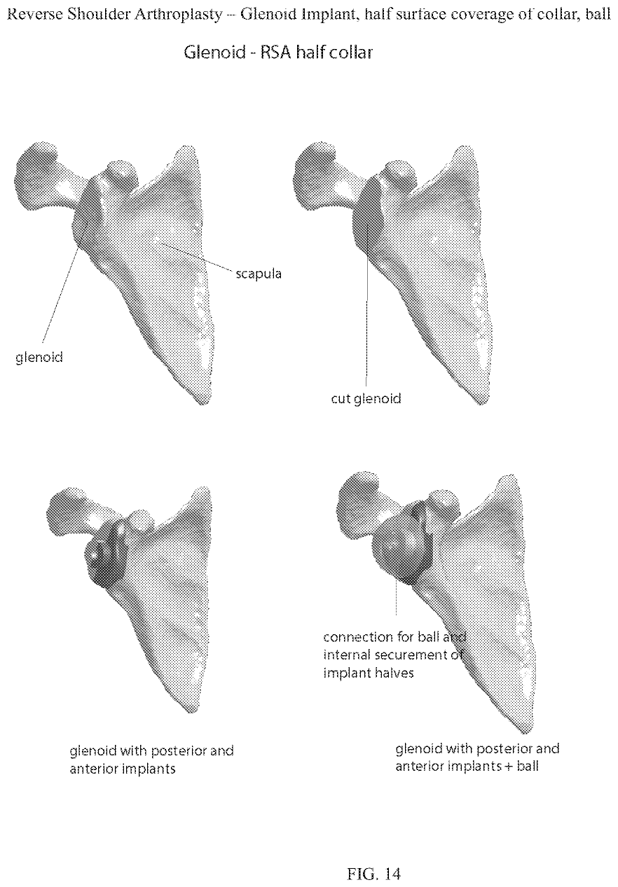

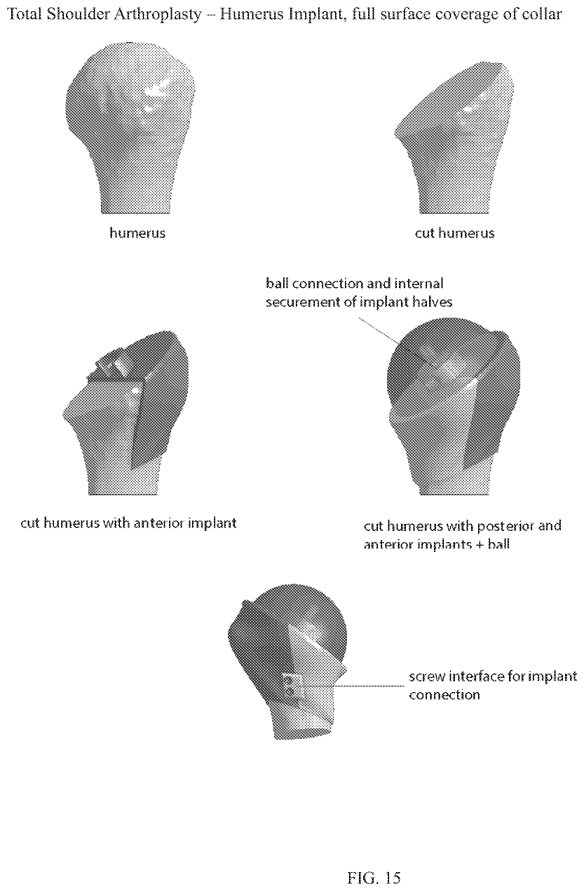

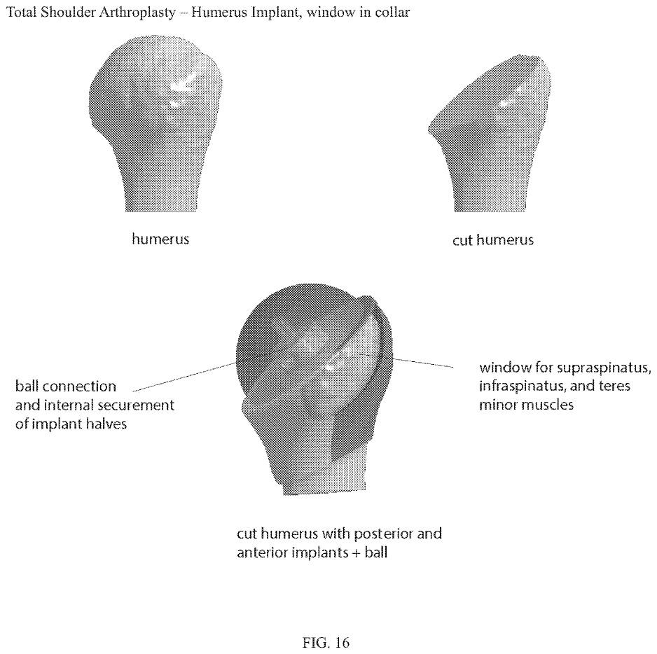

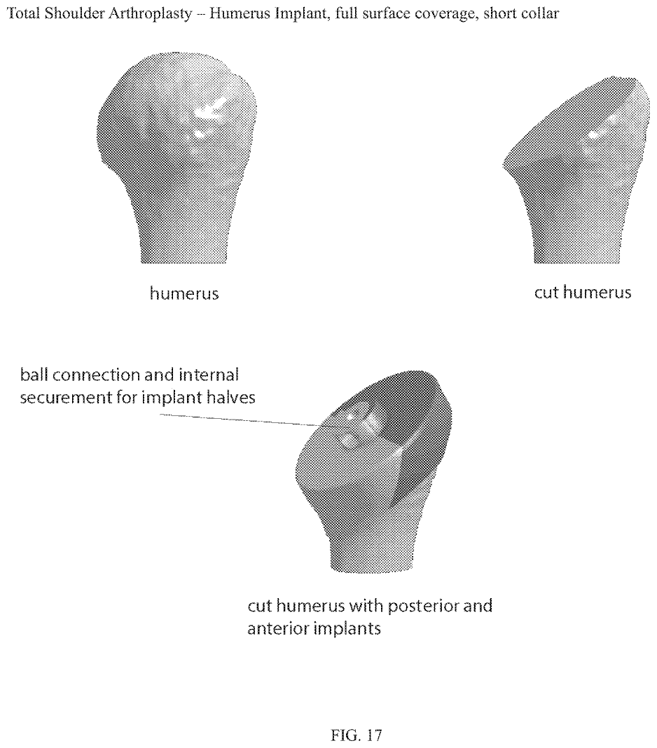

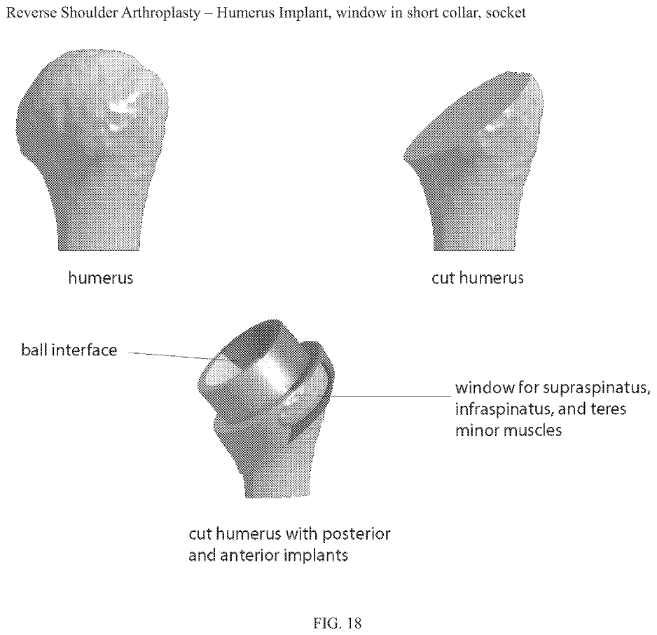

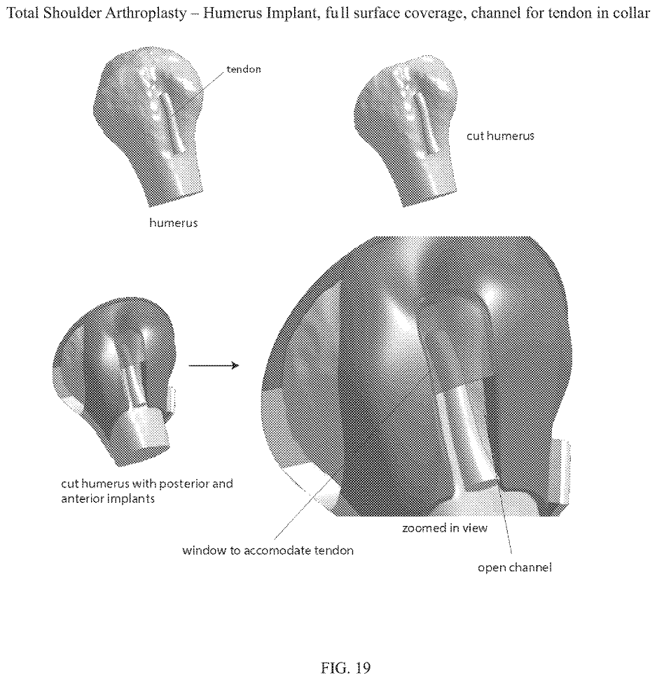

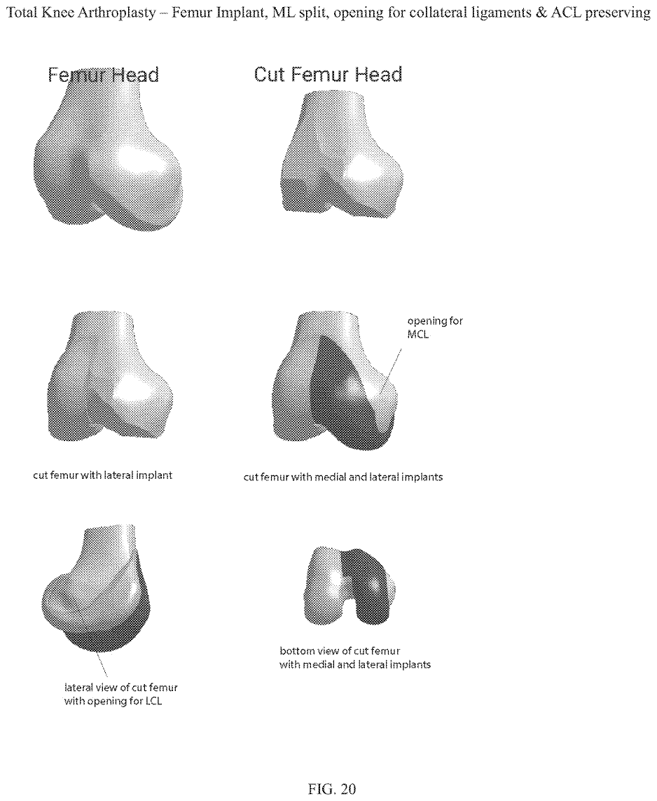

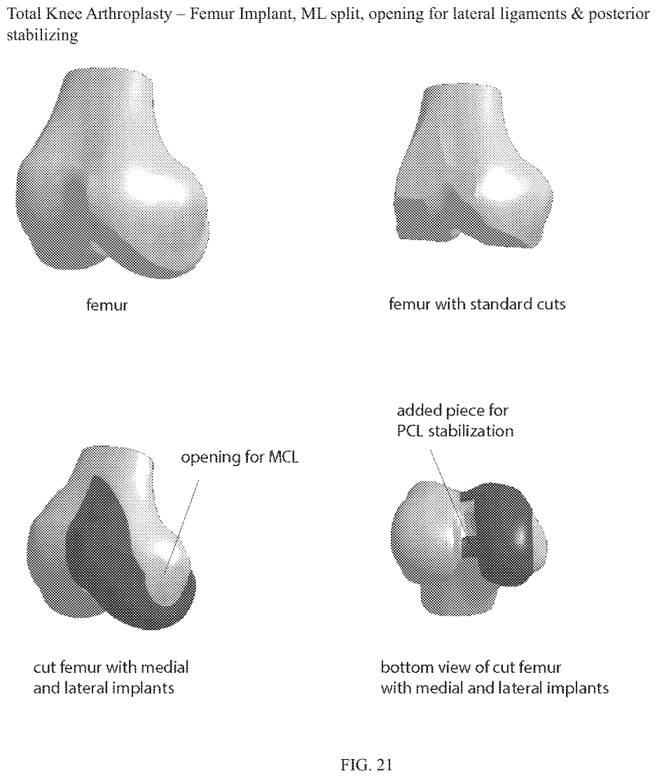

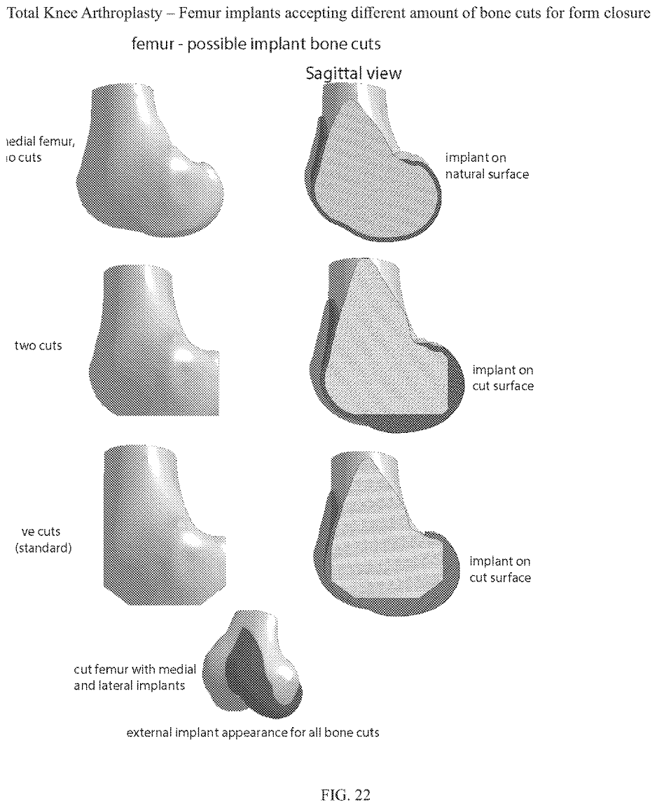

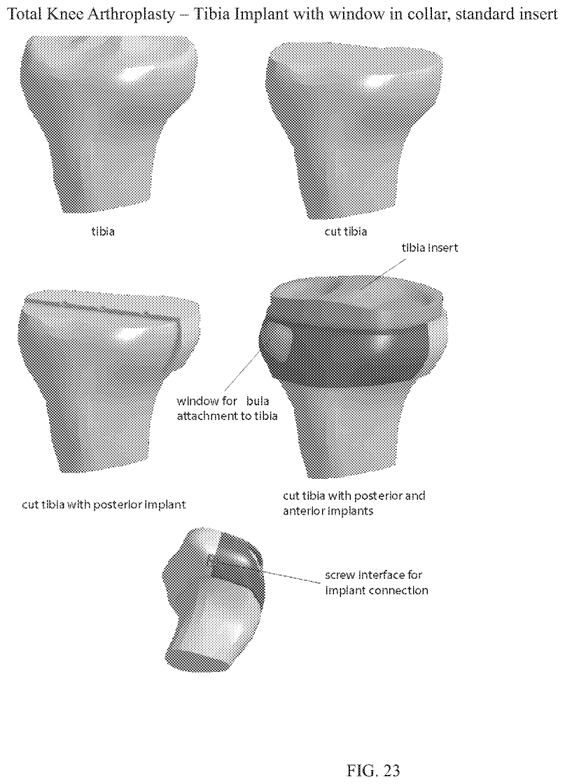

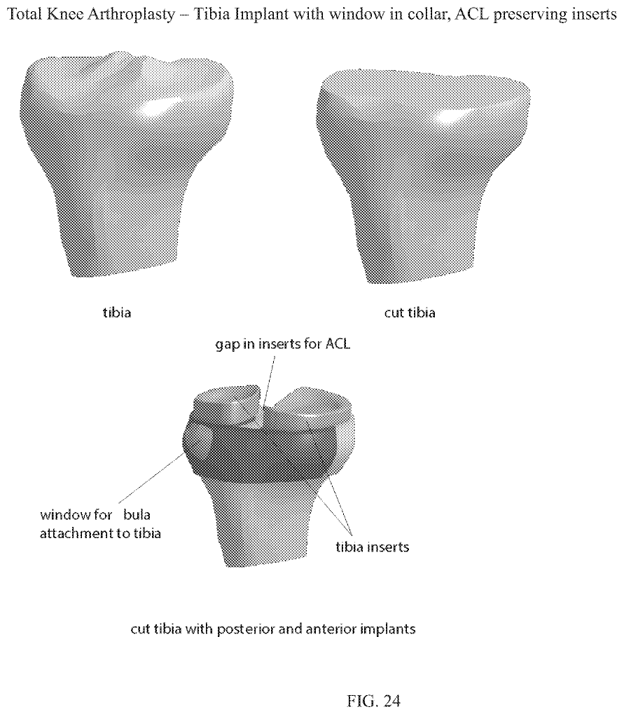

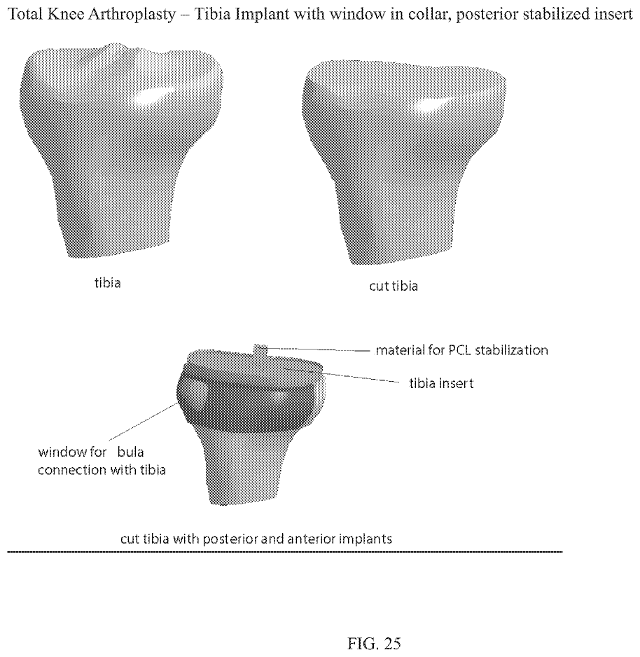

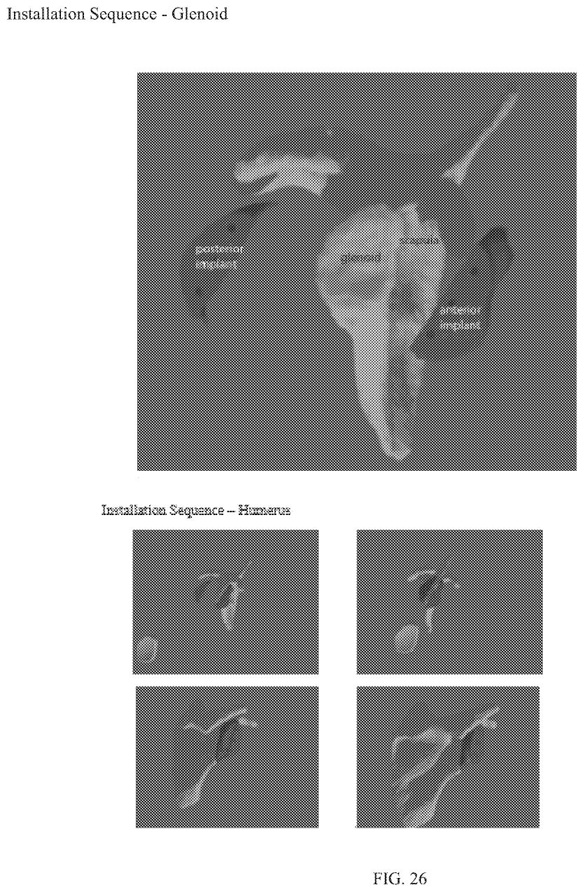

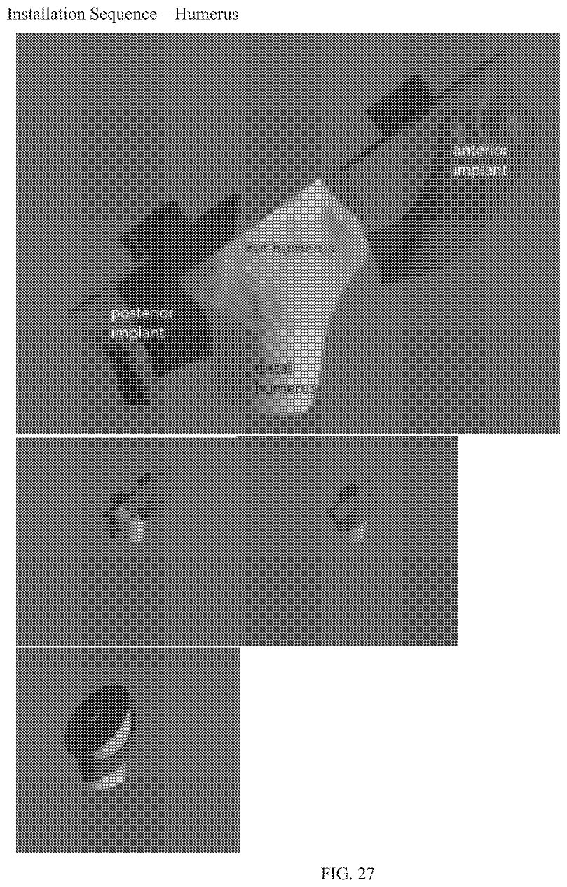

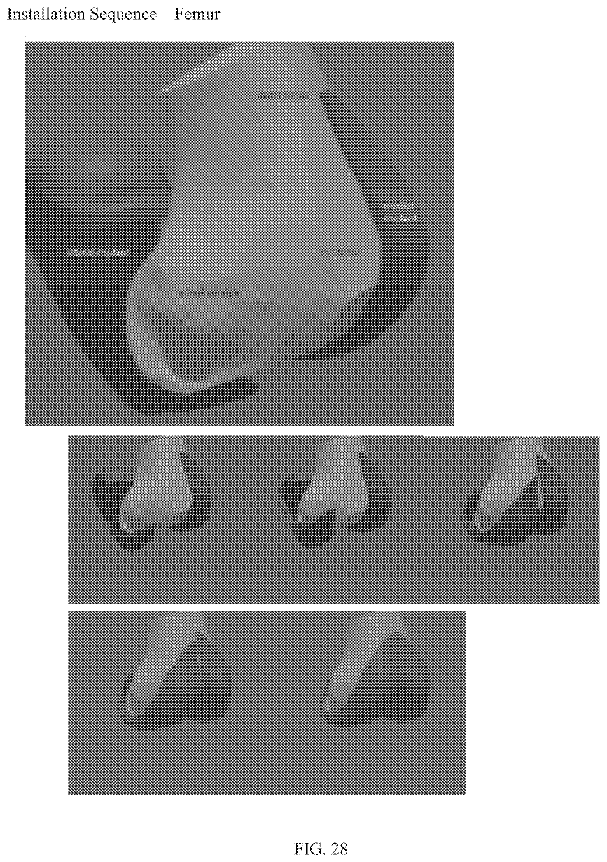

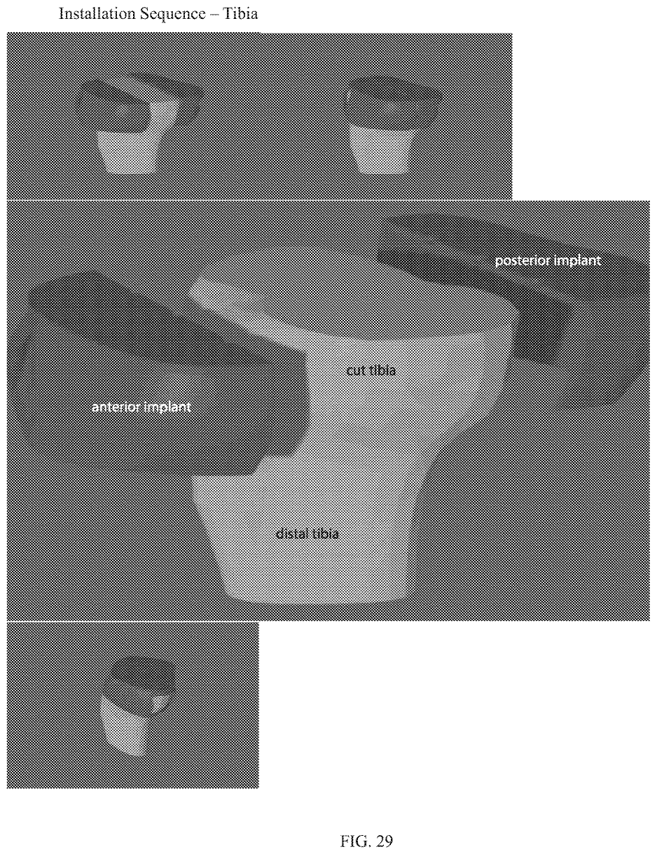

### DETAILED DESCRIPTION

Desiderata for improved joint replacements. To optimize over conventional joint replacements, an approach has been developed for securement that does not require penetrating bolts or stems or bonding agents for its primary mode of mechanical fixation, though these could be used as secondary fixation. The novel method of securement would be easy to install in surgery, would function immediately after surgery so that the patient can resume activities as soon as possible, would provide mechanical securement by distributing force optimally to reduce stress shielding of the bone for optimal dynamics, would provide natural anatomical alignment of implant components to restore optimal kinematics, and would be maximally bone preserving and reversible to allow for ease of future revision if needed. A device satisfying these desirables would improve patient outcomes, and by mitigating the risk/benefit ratio, would appeal to and be adopted by a wider segment of the population.

Complex, non-convex geometry in shoulder and knee. The method of force closure (“firm grip” of the object) by appropriate choice of geometry of contacts (number, location, curvature and extent) where implants enclose the outer surface of bone rather than penetrate it can provide many benefits and is straightforward to apply around convex bones like the cranium. A challenge arises in applying enclosing implants to bone ends which contain surface concavities in addition to surface convexities. The concavities of the glenoid (shoulder), humerus (shoulder and elbow), and distal end of femur (knee) all present additional local surface geometry to consider in enclosing/encasing implant designs. Each of these joints also has its own idiosyncratic points of attachment to soft tissue that need to be preserved by any overlying enclosing implant design. Thus, force closure around the local surface geometry needs to ideally avoid, or provide clearance for, local landmarks while also accounting for complex contact geometries where curvature sign varies from convex to concave, requiring novel installation approaches.

Force closure. When grasping a rigid object such as bone, all degrees of freedom in translation and rotation can be constrained with a sufficient number of touch points and forces, also termed force closure (“firm grip” of the object), which is a general case of form closure that purely relies on touch point geometry for “firm grip” and not additional forces like friction. The force closure method corresponds to enclosing the intrinsic geometry of the available external bone surface providing securement without actually penetrating the bone. When a surface is purely convex (positive curvature exceeding 180 degrees in total), then force closure can easily be achieved by a two-part device that clamps around the rounded geometry such that at least 180 degrees of curvature is traversed in a given plane. Examples of convex geometry include a sphere (constant curvature) or ellipsoid (smoothly varying positive curvature) which approximates the cranium, for example.

Force closure for surfaces containing concavities. By definition, all closed, bounded objects such as a bone end, often corresponding to the bone epiphysis, contain convexities; however, more complex surfaces can also feature intervening concavities. This presents both challenges and opportunities for use of force closure around the mixed convex/concave surface. A benefit is that concavities increase the available surface area for mechanical purchase in increasing the surface features available for grasping the object via enclosure. For example, considering force closure in one 2D plane, an implant can enclose any local region of the undulating surface that admits greater than 180 degrees of curvature locally. Thus, the implant need not surround the global extent of the surface with such a local strategy. Alternatively, the implant can adhere to the global, convex hull to enclose the bone end at the risk of ignoring local geometry, or to be even less constraining, to a set of well spaced external touchpoints can be used for securement globally without interacting with local geometry such as surface undulations. If a force closure geometry is chosen to be inclusive of the concavity features of a surface, novel challenges emerge in installation of the implant parts. In particular, the added surface features (undulations) can interfere with pure sliding or translation of implant pieces over the target bone of interest.

Convex and concave shape of bone ends. Bone ends in the shoulder, knee and elbow do not contain strictly positive curvature and have regions of negative curvature (concavity). In the knee, the distal end of the femur features a concavity or recess between the highly convex lateral and medial epicondyles. In the shoulder, the bicipital groove containing the biceps tendon is a concave feature or ravine in the proximal end (head) of the humerus. Also in the shoulder, the uniquely shaped glenoid contains mild concavities in the perimeter between the infraglenoid and supraglenoid tubercles.

Force closure implants accommodate cuts in bone ends. Often cuts are introduced into the bone end to remove compromised bone/cartilage as in artificial joint replacements (arthroplasty). These cuts effectively change the local curvature to make what was once curved be a flat planar surface (zero curvature on the interior) but creating high curvature corners or edges at the perimeter of the cut. Topologically speaking, the new surface still retains its overall global convexity; therefore, the same principles of force closure apply after introduction of cut ends. Indeed, our method for making enclosing collars can make cutting more precise by using 3D printed guides where a conforming collar wraps the bone portion to be preserved indicating the line/plane above which the cut can be made. In standard arthroplasty procedures, cuts are made largely in a similar manner and location across patients. The disclosed implant embodiments demonstrate designs conforming to prepared bone featuring a combination of natural and cut geometry. The general principle of the invention's function is ultimately agnostic to choice of cuts and idiosyncrasies of patient local encountered in conventional arthroplasty procedures. In other words, there is some discretion of the surgeon to perform more cuts or bone removal, and the implant can still be fitted to conform to the new locally modified geometry while maintaining rigid force closure overall. An example of implant compatibility with modified geometry is the knee joint, where often multiple small planar cuts are introduced into the distal end of the femur that effectively retain the globally curved structure of the epicondyles. In other locations like the proximal end of the tibia, only a single large flat cut is introduced, and the implant conforms to this and the remaining natural surface structure at the tibial epiphysis.

Shape-specific implants do not have to be patient-specific. Rather than 3D print implants that conform to the patient's specific bone shape and thus require unique implants for each procedure, the surgeon can choose to modify the bone more completely to attain a standard geometry across patients such that standard, off-the-shelf force closure implants can generally be used across patients. For making cuts, 3D printed cutting guides that might be patient specific and printed out of cheaper, faster materials like resin could allow for precision location of cuts that transform the bone end to a standard geometry compatible with standard versions of the implant. Thus, two strategies are possible for force or form closure—shaping the bone of each patient for a given implant versus shaping the implant for a given patient's bone. A more fully standardized (patient non-specific) generic shape based method may be imminently usable in knee arthroplasty where multiple cuts are often made around the epicondyles to utilize non-patient specific standard implant pieces that overlay the cut distal femur bone epiphysis.

Concave bone regions allow achieving force closure locally. We take advantage of local concavity in the glenoid, femur, tibia, and humerus to design implants which use local geometry to constrain implant translation and rotation in all degrees of freedom relative to the bone. These sufficient local constraints relax the need for fully enclosing global implants of the bone end surface. For the distal femur, this local securement strategy clears space for the medial and lateral collateral ligaments on the outer aspects of the condyles (the epicondyles), while also leaving space internally between the condyles for the anterior or posterior cruciate ligaments. In the glenoid, in some embodiments securing the implant to the anterior and posterior concavities of the glenoid neck leaves clearance in the dorsal supraglenoid and ventral infraglenoid tubercles for the origins of the biceps brachii and triceps brachii, respectively. In the proximal humerus, an implant invaginating the convex-concave-convex geometry formed by the greater tubercle (convex), intertubercular groove (concave), and lesser tubercle (convex) on the anterior aspect of the neck allows for force closure on local geometry such that implant clearance is possible on the posterior aspect of the neck for insertions of the supraspinatus, infraspinatus, and teres minor muscles.

Installation of implants over bones with irregular curvature using in-plane translation. For purely convex geometries, sliding two implant halves around the object, whether a sphere or an ellipse, is adequate for reaching closure without interference from intervening surface structure in sliding the implant over bone. However, translation of implant pieces will often encounter interferences on the translation path for surfaces with varying curvature sign (i.e., convex and concave). In the case of a convexity followed by a concavity, the component of the implant conforming to the concavity will naturally interfere along certain axes of translation that are aligned or partially aligned with the axis along which curvature sign is changing. In these cases, where one axis contains most curvature variation, then a practical solution is to use implant pieces that internally traverse the complex curves within their component geometry but then install the pieces by sliding along an axis orthogonal to this curvature variation.

Installation of implants over bones with irregular curvature using out-of-plane motion. For complex surfaces where changes in curvature are not constrained to one axis, a method to avoid interferences during installation is to rotate the part out of the plane of locally changing curvature while sliding it into position, after which the part can be rotated back into the correct plane. This method temporarily moves interfering curves out of the way locally. An example where this approach helps is the distal end of the femur, here rotating implants with interfering portions (i.e., high curvature arms overlaying epicondyles) out of plane before sliding over the epicondyle aids the installation. In general, curvature will vary most in certain planes and be flat or at least less varying in other planes for smooth volumes like bone. Thus, the implant installation can be started partially out of plane to allow easier engagement of implant regions within plane without global interference from the out-of-plane portions. Then the remaining implant can be rotated back into plane. In general, some combination of a sequence of in-plane translation/in-plane rotating and concurrent out-of-plane motion will relieve interferences in sliding an enclosing implant over a complex curvature bone surface.

Variations in overall footprint of force closure implants. We list a number of decisions that can be made in the design of the force closure implant, all while still maintaining mechanical fixation. The nature of a distributed full wrap of a geometry based implant fit provides design flexibility because there are many points of contact and only a few are needed to maintain full bone fixation—as few as four contact points could be used to grasp the object's hull, though for biomedical applications, increased bone contact from a continuous surface may be desirable. The extent of the collar can be determined by the desire for trading off implant strength and distribution of force (larger more continuous collar surface for maximal surface purchase) versus minimizing interference with the remaining tissue to be preserved (windows for ligaments or shorter collar) and/or retaining more bone (location of bone cut made higher). The presence of channels or windows in the implant can help with its more seamless integration into the existing joining soft and hard tissue. The force closure can happen via contact to the convex hull of the bone and need not invade concavities where structures to be preserved travel, hence allowing for channels underneath the convex footprint. The advantage of a channel is that it provides overhead clearance for underlying soft tissue such that the tendon or ligament would not need to be detached from bone. In the case of a window, the tendon or ligament may need to be cut and threaded through the window before being reconnected. Such manipulations of soft tissue are done in current procedures to allow clearance, and in some cases tendons or ligaments have their insertions shifted or removed entirely as it does not significantly impact long-term post-op function given compensatory, redundant mechanisms from other muscles. These decisions in reducing touch points, designing windows and/or channels versus entirely removing soft tissue can be easily implemented after consultation with the surgeon and consideration of patient-specific anatomy from medical imaging (i.e., MRI, CT, ultrasound).

Variations in internal securement of force closure implants. Besides choosing the extent of the enclosing implant, the user can choose the method for internal securement of the implant pieces over the bone. Fastening screws can be placed on the outer portion of the collar to connect the two halves of the implant (assuming a two-piece design was used). Alternatively, pieces can be internally secured (e.g., within the shell of a connecting ball portion if used). The securement can be done with screws and/or glues and bonding agents, where now bonding agents are only applied to implant material instead of directly to bone which mitigates biocompatibility issues of cements/glues. Since the pieces interface with each other in securing around the bone, piece-to-piece fixation/securement with glues and bonding agents may be preferred as this could lead to more even force distribution along the seams of implant pieces, and as mentioned these bonding agents would not be directly interfacing with bone which has benefits for biocompatibility. Glues allow for internal securement without having protruding fasteners above the surface of the implant. Finally, penetrating screws could be used to fasten the implant directly to the bone to fix pieces relative to bone and thus relative to each other, in lieu of or in addition to screws for securing implant pieces directly to each other. This may provide additional stability from screws including the potential for osseointegration to the screws. All methods, external fastening between implant pieces by glue or screws, internal fastening between implant pieces by glue or screws, and fastening direct to bone are not mutually exclusive and can function in complementary roles for improving implant securement while managing its overall profile in the body for reducing impingement of overlying soft tissue.

Variation in implant number of parts and seam geometry. In order to enclose via force closure, the implant has to be divided into at least two pieces and the presented embodiments tend to adhere to this minimal—and thus maximally constraining for installation—number of pieces to demonstrate the general feasibility of force closure including for any less constraining design parameters such as dividing the implant into more than two pieces. The user could divide implants into more than two pieces depending on considerations like ease of installation (smaller pieces provide less interference if surgical space is tight) or for locating seams in strategic places. The seam could be considered a potentially mechanically weak fracture line of the overall implant and while the screw/glue securement of the implant pieces described in the previous section can be optimized to strengthen this interface, one can also consider seam geometries other than line geometries. We suggest an interlocking design will potentially redirect force into two dimensions at the seam rather than be susceptible to sliding/shearing force along the seam's length. Other local seam shapes for improved locking of pieces could be implemented such as over-under dovetails. These design choices for the seam interface would need to adhere to the overall path of installation of the force closure implant. If the installation path goes on an axis not aligned with the presence of features like interlocking teeth, then it will be hard to slide the implant fully closed.

Other treatments for improving implant-bone adherence. The enclosing implant method does not preclude combining with existing methods for improving osseointegration such as applying surface coatings to the implant, scoring of the interior surface of the implant to provide local surface features for bone in-growth, using porous titanium or other biocompatible material that favors bone integration, or using small penetrating screws in implant regions for added bone integration. Besides primary securement via force closure of the implant pieces over the bone, screws or cement further securing implant into bone can be used as complementary secondary forms of securement for added general strength, for locally redirecting forces into bone as seems optimal, or entirely as backup securement measures for long-term life of the implant should bone degrade and compromise the quality of primary force closure based securement to any degree.

### Aspects

The present disclosure provides prostheses and methods according to any of the following aspects. Any part or parts of any Aspect can be combined with any part or parts of any other Aspect.

Aspect 1. A prosthesis for securement to an end portion of a bone that includes both positive and negative curvature, comprising: a first enclosure component that at least partially conforms to and engages a first portion of a side surface of the bone and a first portion of an end surface of the bone; a second enclosure component that at least partially conforms to and engages a second portion of a side surface of the bone and a second portion of an end surface of the bone, the first portion of the side surface of the bone being on a substantially opposing side of the bone as the second portion of the side surface of the bone, and wherein the first enclosure component is configured to be coupled to the second enclosure component while said first and second enclosure components are positioned on the bone in order to provide mechanical securement of the prosthesis to the bone.

Aspect 2. The prosthesis according to Aspect 1, comprising an end surface that is defined by the engagement of the first enclosure component with the second enclosure component at the end surface of the bone.

Aspect 3. The prosthesis according to Aspect 2, wherein the prosthesis further comprises a ball portion or a cup portion that is configured to be coupled to the end surface.

Aspect 4. The prosthesis according to Aspect 3, wherein the prosthesis comprises the ball portion, and the ball portion defines a concave surface that is configured to engage with a concave surface of a further bone at an anatomical joint.

Aspect 5. The prosthesis according to Aspect 3, wherein the prosthesis comprises the cup portion, and the cup portion defines one or more concavities that are each configured to engage with a convex surface of a further bone at an anatomical joint.

Aspect 6. The prosthesis according to any preceding Aspect, wherein the first enclosure component, the second enclosure component, or both, comprises a window or a channel that is configured to spatially accommodate an anatomical feature other than a bone of a subject.

Aspect 7. The prosthesis according to any preceding Aspect, wherein said first enclosure portion and said second enclosure portion are configured for being coupled together using a screw, bolt, adhesive, or by interlocking respective parts of said first enclosure portion and said second enclosure portion.

Aspect 8. The prosthesis according to any preceding Aspect, wherein the prosthesis is configured for implantation onto an end of a bone that has been cut in at least one location prior to implantation of the prosthesis.

Aspect 9. The prosthesis according to any preceding Aspect, further comprising one or more additional enclosure components that are respectively configured to be coupled to at least one other enclosure components while positioned on the bone in order to provide mechanical securement of the prosthesis to the bone.

Aspect 10. The prosthesis according to any preceding Aspect, wherein said prosthesis (1) is configured for implantation onto a glenoid cavity of a subject, and (2) comprises either a cup portion defining a concavity that is configured to engage with a convex surface of the subject's humerus pursuant to a total shoulder arthroplasty procedure, or a ball portion that is configured to engage with a concave surface of the subject's humerus pursuant to a reverse shoulder arthroplasty procedure.

Aspect 11. The prosthesis according to any one of Aspects 1-9 that is (1) configured for implantation onto and end of the subject's humerus, and (2) comprises either a ball portion that is configured to engage with a concave surface of the subject's glenoid pursuant to a total shoulder arthroplasty procedure, or a cup portion defining a concavity that is configured to engage with an artificial convex surface of the subject's glenoid pursuant to a reverse shoulder arthroplasty procedure.

Aspect 12. The prosthesis according to any one of Aspects 1-9 that is (1) configured for implantation onto and end of the subject's tibia, and (2) comprises either a ball portion that is configured to engage with a concave surface of the subject's femur pursuant to a total knee arthroplasty procedure, or a cup portion that is configured to engage with an artificial convex surface of the subject's femur pursuant to a reverse knee arthroplasty procedure.

Aspect 13. The prosthesis according to any one of Aspects 1-9 that is (1) configured for implantation onto and end of the subject's femur, and (2) comprises either a ball portion that is configured to engage with a concave surface of the subject's hip pursuant to a total hip arthroplasty procedure, or a cup portion that is configured to engage with an artificial convex surface of the subject's hip pursuant to a reverse hip arthroplasty procedure.

Aspect 14. A method for securing a prothesis according to any one of the preceding Aspect to an end of a bone, comprising: using a jig or guide to determine a cut location on the end of the bone; cutting the bone at the cut location to form a cut section of the bone; engaging at least a portion of the cut section of the bone with the first enclosure portion of the prosthesis; installing the second enclosure portion of the prosthesis onto the end of the bone; and, coupling the first enclosure portion and the second enclosure portion, thereby securing the prosthesis to the end of the bone.

What is claimed:

1. A prosthesis for securement to an end portion of a bone that includes both positive and negative curvature, comprising:
a first enclosure component that at least partially conforms to and engages a first portion of a side surface of the bone and a first portion of an end surface of the bone;
a second enclosure component that at least partially conforms to and engages a second portion of a side surface of the bone and a second portion of an end surface of the bone,
the first portion of the side surface of the bone being on a substantially opposing side of the bone as the second portion of the side surface of the bone, and
wherein the first enclosure component is configured to be coupled to the second enclosure component while said first and second enclosure components are positioned on the bone in order to provide mechanical securement of the prosthesis to the bone.

2. The prosthesis according to claim 1, comprising an end surface that is defined by the engagement of the first enclosure component with the second enclosure component at the end surface of the bone.

3. The prosthesis according to claim 2, wherein the prosthesis further comprises a ball portion or a cup portion that is configured to be coupled to the end surface.

4. The prosthesis according to claim 3, wherein the prosthesis comprises the ball portion, and the ball portion defines a concave surface that is configured to engage with a concave surface of a further bone at an anatomical joint.

5. The prosthesis according to claim 3, wherein the prosthesis comprises the cup portion, and the cup portion defines one or more concavities that are each configured to engage with a convex surface of a further bone at an anatomical joint.

6. The prosthesis according to claim 1, wherein the first enclosure component, the second enclosure component, or both, comprises a window or a channel that is configured to spatially accommodate an anatomical feature other than a bone of a subject.

7. The prosthesis according to claim 1, wherein said first enclosure portion and said second enclosure portion are configured for being coupled together using a screw, bolt, adhesive, or by interlocking respective parts of said first enclosure portion and said second enclosure portion.

8. The prosthesis according to claim 1, wherein the prosthesis is configured for implantation onto an end of a bone that has been cut in at least one location prior to implantation of the prosthesis.

9. The prosthesis according to claim 1, further comprising one or more additional enclosure components that are respectively configured to be coupled to at least one other enclosure components while positioned on the bone in order to provide mechanical securement of the prosthesis to the bone.

10. The prosthesis according to claim 1, wherein said prosthesis (1) is configured for implantation onto a glenoid cavity of a subject, and (2) comprises either a cup portion defining a concavity that is configured to engage with a convex surface of the subject's humerus pursuant to a total shoulder arthroplasty procedure, or a ball portion that is configured to engage with a concave surface of the subject's humerus pursuant to a reverse shoulder arthroplasty procedure.

11. The prosthesis according to claim 1 that is (1) configured for implantation onto and end of the subject's humerus, and (2) comprises either a ball portion that is configured to engage with a concave surface of the subject's glenoid pursuant to a total shoulder arthroplasty procedure, or a cup portion defining a concavity that is configured to engage with an artificial convex surface of the subject's glenoid pursuant to a reverse shoulder arthroplasty procedure.

12. The prosthesis according to claim 1 that is (1) configured for implantation onto and end of the subject's tibia, and (2) comprises either a ball portion that is configured to engage with a concave surface of the subject's femur pursuant to a total knee arthroplasty procedure, or a cup portion that is configured to engage with an artificial convex surface of the subject's femur pursuant to a reverse knee arthroplasty procedure.

13. The prosthesis according to claim 1 that is (1) configured for implantation onto and end of the subject's femur, and (2) comprises either a ball portion that is configured to engage with a concave surface of the subject's hip pursuant to a total hip arthroplasty procedure, or a cup portion that is configured to engage with an artificial convex surface of the subject's hip pursuant to a reverse hip arthroplasty procedure.

14. A method for securing a prothesis according to claim 1 to an end of a bone, comprising:
using a jig or guide to determine a cut location on the end of the bone;
cutting the bone at the cut location to form a cut section of the bone;
engaging at least a portion of the cut section of the bone with the first enclosure portion of the prosthesis;
installing the second enclosure portion of the prosthesis onto the end of the bone; and,
coupling the first enclosure portion and the second enclosure portion, thereby securing the prosthesis to the end of the bone.
